(19) United States
(12) Patent Application Publication
(10) Pub. No.: US 2018/0014808 A1
(43) Pub. Date: Jan. 18, 2018

(54) X-RAY CT APPARATUS AND SCANNING METHOD

(71) Applicant: HITACHI, LTD., Tokyo (JP)

(72) Inventor: Motoshi MASUDA, Tokyo (JP)

(21) Appl. No.: 15/547,832

(22) PCT Filed: Mar. 8, 2016
(86) PCT No.: PCT/JP2016/057111

§ 371 Date: Aug. 1, 2017

(30) Foreign Application Priority Data
Mar. 31, 2015 (JP) 2015-072373

(51) Int. Cl. A61B 6/00; A61B 6/03

(52) U.S. Cl. CPC A61B 6/54; A61B 6/032

(57) ABSTRACT

In order to provide an X-ray CT apparatus and a scanning method, capable of improving examination throughput, and suppressing wearing of a bearing of a rotation disk, a system control device 124 of an X-ray CT apparatus 1 determines whether or not a rotation speed control operation can be performed for a standby time on the basis of an acceleration time and a deceleration time to respective rotation speeds Sₙ and Sₘ in successive scanning operations, and a standby interval time Tᵢₙₜ, determined according to scanning conditions, decelerates the rotation disk 102 to a target low rotation speed or a rotation stoppage state if the rotation speed control operation can be performed, waits until a predetermined standby time T_{Idle} elapses at the target low rotation speed or in the rotation stoppage state, and then increases a rotation speed of the rotation disk to a rotation speed in the next sequence.

### TECHNICAL FIELD

The present invention relates to an X-ray CT apparatus and a scanning method, and particularly to control of a rotation speed of a scanner of an X-ray CT apparatus.

### BACKGROUND ART

An X-ray CT apparatus includes aa X-ray source which irradiates an object with X-rays, and an X-ray detector which detects a dose of X-rays transmitted through the object as projection data, and creates a tomographic image of the object by using projection data from a plurality of angles obtained by rotating the X-ray source and the X-ray detector around the object. In scanning using the X-ray CT apparatus, it is necessary to increase a rotation speed of a scanner in order to perform scanning on a region requiring a high temporal resolution or to reduce scanning time. Thus, regarding a rotation speed of a rotation disk of the X-ray CT apparatus, the time required for the rotation speed to be constant from starting of acceleration is increased, and this is one of factors of reducing examination throughput.

PTL 1 discloses an X-ray CT apparatus which detects finishing of capturing of a scanogram image, and performs control so that rotation of a rotation disk is started so as to reduce the time from scanning preparation to starting of main scanning.

### CITATION LIST

### Patent Literature

PTL 1: JP-A-2010-124924

### SUMMARY OF INVENTION

### Technical Problem

However, in a case of an examination such as a contrast examination in which standby time (hereinafter, referred to as inter-sequence delay time) between scanning operations is set to be long, rotation of a rotation disk is maintained in an X-ray CT apparatus of the related art for the inter-sequence delay time. Since the rotation disk is rotatably supported with respect to a fixation portion of a scanner via a bearing, wearing of the bearing progresses due to an increase in rotation time, and this influences the durability of the apparatus.

The invention has been made in consideration of the above-described problem, and an object thereof is to provide an X-ray CT apparatus and a scanning method, capable of improving examination throughput, and suppressing wearing of a bearing of a rotation disk.

### Solution to Problem

In order to achieve the above-described object, according to the invention, there is provided an X-ray CT apparatus including a scanner that includes an X-ray source which irradiates an object with X-rays, an X-ray detector which is disposed to oppose the X-ray source and detects X-rays transmitted through the object, and a rotation disk that is mounted with the X-ray source and the X-ray detector and is rotated around the object; and a controller that controls a rotation speed of the rotation disk for a standby time to be lower than all rotation speeds in successive scanning operations on the basis of the standby time between the successive scanning operations and the times required for deceleration and acceleration of the rotation disk.

According to the invention, there is provided a scanning method for an X-ray CT apparatus including an X-ray source which irradiates an object with X-rays, an X-ray detector which is disposed to oppose the X-ray source and detects X-rays transmitted through the object, and a rotation disk that is mounted with the X-ray source and the X-ray detector and is rotated around the object, the method including causing a controller of the X-ray CT apparatus to control a rotation speed of the rotation disk for a standby time to be lower than all rotation speeds in successive scanning operations on the basis of the standby time between the successive scanning operations and the times required for deceleration and acceleration of the rotation disk.

### Advantageous Effects of Invention

According to the invention, it is possible to provide an X-ray CT apparatus and a scanning method, capable of improving examination throughput, and suppressing wearing of a bearing of a rotation disk.

### BRIEF DESCRIPTION OF DRAWINGS

FIG. 1 is a diagram illustrating the entire configuration of an X-ray CT apparatus 1.

FIG. 2 is a flowchart illustrating a flow of a rotation speed control process.

FIG. 3 is a timing chart in a case where a rotation speed is zero for inter-sequence delay time (standby time between successive scanning operations).

FIG. 4 is a timing chart in a case where a rotation speed is maintained to be as low as possible for inter-sequence delay time.

FIG. 5 is a flowchart in a case where a rotation speed control operation is -started before a scanning starting operation is detected.

FIG. 6 is a timing chart illustrating an operation corresponding to the flowchart in FIG. 5.

### DESCRIPTION OF EMBODIMENTS

Hereinafter, with reference to the drawings, embodiments of the invention will be described in detail.

### First Embodiment

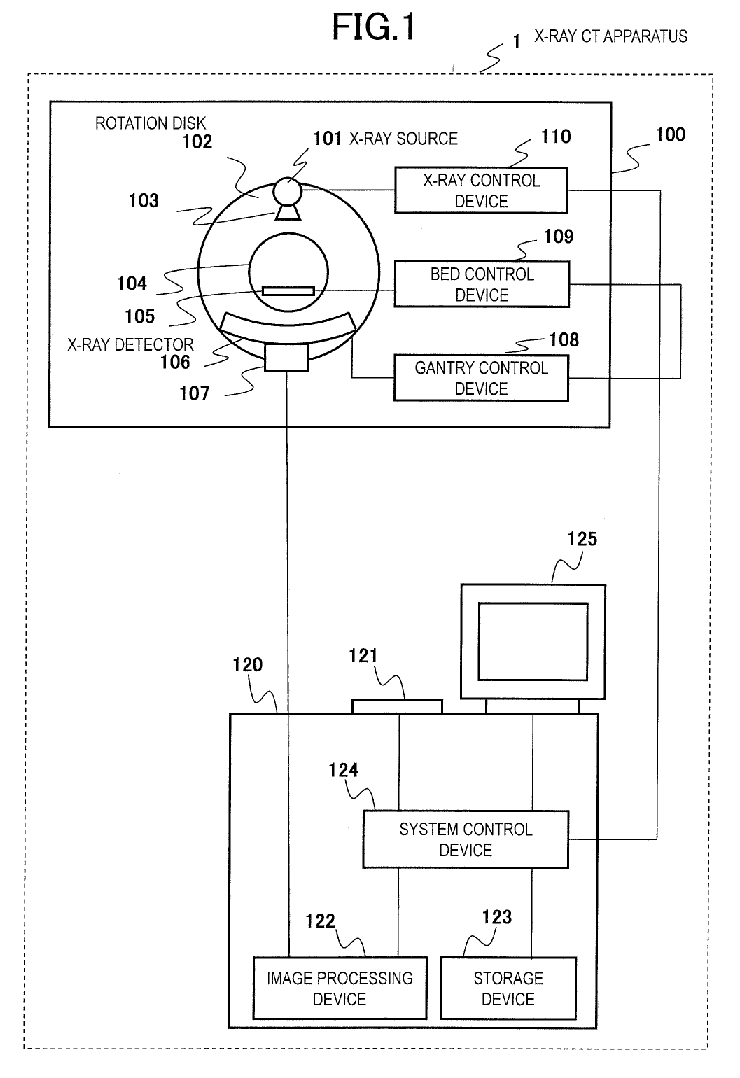

First, a description will be made of the entire configuration of an X-ray CT apparatus 1 with reference to FIG. 1.

As illustrated in FIG. 1, the X-ray CT apparatus 1 includes a scanner 100, a bed 105, and an operation console 120. The scanner 100 is a device which irradiates an object with X-rays and detects X-rays transmitted through the object. The operation console 120 is a device which controls each constituent element of the scanner 100, and acquires transmitted X-ray data measured by the scanner 100 so as to generate an image. The bed 105 is a device on which the object is laid and is mounted and which carries the object into and out of an X-ray irradiation range of the scanner 100.

The scanner 100 includes an X-ray source 101, a rotation disk 102, a collimator 103, an X-ray detector 106, a data collecting device 107, a gantry control device 108, a bed control device 109, and an X-ray control device 110.

The operation console 120 includes an input device 121, an image calculation device 122, a storage device 123, a system control device 124, and a display device 125.

The rotation disk 102 of the scanner 100 is provided with an opening 104, and the X-ray source 101 and the X-ray detector 106 are disposed to oppose each other with the opening 104 interposed therebetween. An object, mounted on the bed 105 is inserted into the opening 104. The rotation disk 102 is rotated around the object by a driving force which is transmitted from a rotation disk driving device via a driving transmission system. The rotation disk driving device is controlled by the gantry control device 108.

The X-ray source 101 is controlled by the X-ray control device 110 so as to apply X-rays with a predetermined intensity continuously or intermittently. The X-ray control device 110 controls an X-ray tube voltage applied or supplied to the X-ray source 101 and an X-ray tube current supplied thereto according to an X-ray tube voltage and an X-ray tube current determined by the system control device 124 of the operation console 120.

The collimator 103 is provided in an X-ray irradiation outlet of the X-ray source 101. The collimator 103 restricts an irradiation range of X-rays applied from the X-ray source 101. For example, an irradiation range is shaped into a cone beam (conical or pyramidal beam). An aperture width of the collimator 103 is controlled by the system control device 124.

The X-rays, applied from the X-ray source 101, passing through the collimator 103, and transmitted through the object, are incident on the X-ray detector 106.

The X-ray detector 106 is a detector in which, for example, X-ray detection element groups each formed of a scintillator and a photodiode are two -dimensionally arranged in a channel direction (rotation direction) and a column direction (body axis direction). The X-ray detector 106 is disposed to oppose the X-ray source 101 via the object. The X-ray detector 106 detects a dose of X-rays applied from the X-ray source 101 and transmitted through the object, and outputs the dose to the data collecting device 107.

The data collecting device 107 collects an X-ray dose detected by each X-ray detection element of the X-ray detector 106, converts the X-ray close into a digital signal, and sequentially outputs the digital signal to the image calculation device 122 of the operation console 120 as transmitted X-ray data.

The image calculation device 122 is a computer provided with a central processing unit (CPU), a read only memory (ROM), a random access memory (RAM), and the like. The image calculation device 122 acquires the transmitted X-ray data which is input from the data collecting device 107, and performs pre-processing such as logarithmic conversion and sensitivity correction on the transmitted X-ray data so as to generate projection data which is necessary in reconstruction. The image calculation device 122 reconstructs an image such as a tomographic image drawing the inside of the object by using the generated projection data. The system control device 124 stores the image data reconstructed by the image calculation device 122 in the storage device 123, and also displays the image data on the display device 125.

The system control device 124 is a computer provided with a CPU, a ROM, a RAM, and the like.

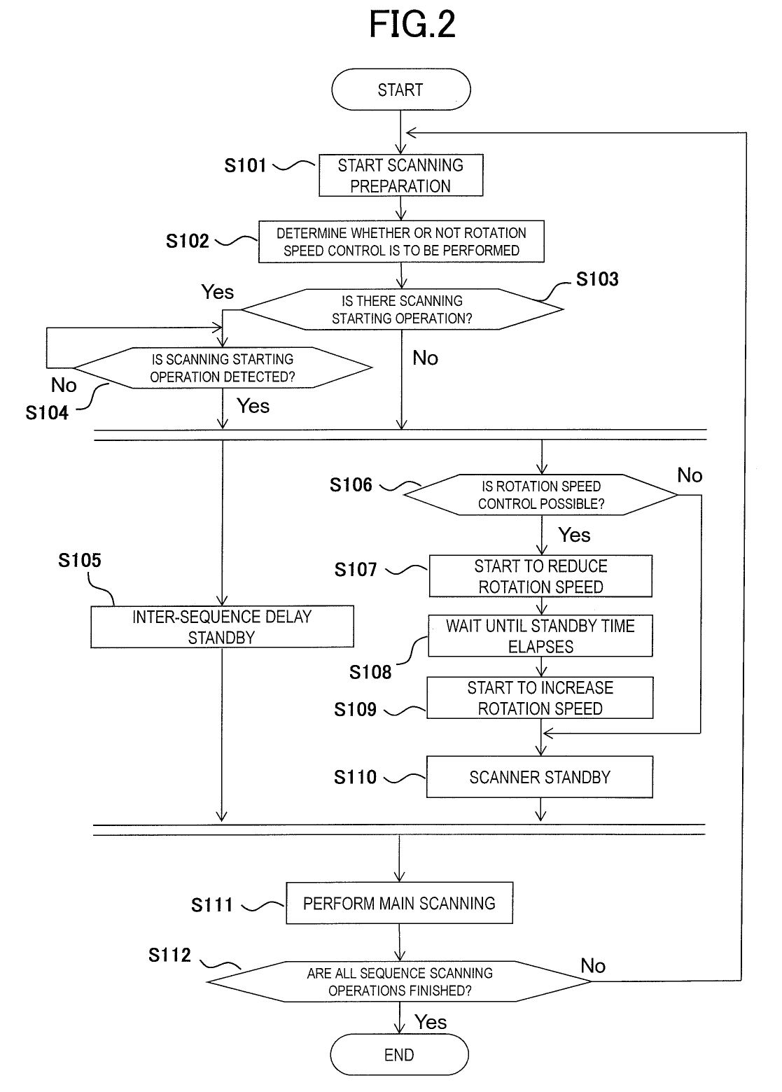

In the invention, the system control device 124 performs a rotation speed control process illustrated in FIG. 2 or the like according to a process program stored in the storage device 123 or the ROM. In the rotation speed control process, the system, control device 124 controls a rotation speed of the rotation disk 102 for a standby time between successive scanning operations (inter-sequence delay time). The rotation speed control process will be described later.

The storage device 123 is a data recording device such as a hard disk, and stores in advance programs or data for realizing a function of the X-ray CT apparatus 1. Such program codes are read to the RAM as necessary by the system control device 124, and are then read to the CPU so as to be executed.

The display device 125 is constituted of a display device such as a liquid crystal panel or a CRT monitor, and a logic circuit for performing a display process in conjunction with the display device, and is connected to the system control device 124. The display device 125 displays an image output from the image calculation device 122, and various pieces of information treated by the system control device 124.

The input device 121 is formed of, for example a pointing device such as a keyboard or a mouse, and various switch buttons, and outputs various instructions or information input by an operator, to the system control device 124. An operator operates the X-ray CT apparatus 1 in an interaction manner by using the display device 125 and the input device 121. The input device 121 may be a touch panel type input device which is integrally formed with a display screen of the display device 125.

The bed 105 includes a top plate on which an object is laid and mounted, a vertical movement device, and a top plate driving device. The top plate is moved up and down, moved in a front-and-rear direction along the body axis direction, or moved in a leftward-and-rightward direction which is perpendicular to a body axis and is parallel to a floor surface, under the control of the bed control device 109. During scanning, the bed control device 109 moves the top plate at a bed movement, speed and in a movement direction determined by the system control device 124.

Next, with reference to a flowchart in FIG. 2, a description will be made a flow of a rotation speed control process performed by the system control device 124 of the first embodiment. The system control device 124 reads a rotation speed control process program from the storage device 123 or the ROM, and performs a rotation speed control process illustrated in FIG. 2 according to the process program.

First, the system control device 124 starts scanning preparation (step S101). In step S101, the system control device 124 transmits control signals to the X-ray control device 110, the gantry control device 108, and the bed control device 109 on the basis of scanning conditions which are set in advance by an operator using the input device 121, and starts scanning preparation. The scanning conditions include X-ray conditions such as an X-ray tube voltage or an X-ray tube current, a rotation speed of the rotation disk 102, a spiral pitch, and the like, and are input and set via the input device 121 by the operator. It is assumed that various parameters such as a length of the inter-sequence delay time, a lower limit value Tₜₕ or the like of standby time in a rotation stoppage state or a deceleration rotation state which will be described later, used for the rotation speed control process are set in advance before starting a rotation control operation.

The inter-sequence delay time is, for example, a time until X-ray irradiation is actually started after the operator performs, for example, a scanning starting operation such as pressing of an X-ray irradiation starting button, or a time from finishing of X-ray irradiation in the previous scanning (sequence) to starting of X-ray irradiation in the next scanning (sequence). Whether or not a scanning starting operation performed by the operator is received is assumed to be set in advance. A length of the inter-sequence delay time may be set to any value by the operator in advance.

Next, the system, control device 124 determines whether or not rotation speed control is to be performed (step S102). In step S102, the system control device 124 computes a standby interval time T_{Int} on the basis of the set inter-sequence delay time. The standby interval time T_{Int} is a time period from a start time point (that is, at the time at which X-ray irradiation is finished in the previous scanning (sequence), or when the operator performs a scanning starting operation) of the inter-sequence delay time to a time point at which scanner standby is started. The system control device 124 acquires a set value of a target, low rotation speed or the like which is set in advance.

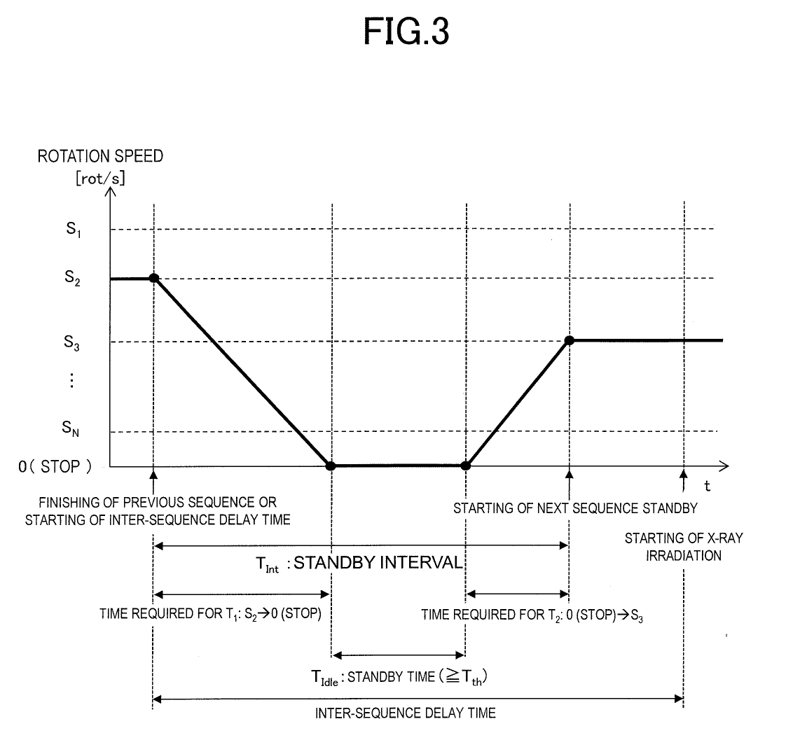

The system control device 124 determines whether or not a rotation speed control operation can be performed for the standby time (inter-sequence delay time) between two successive sequences (scanning) on the basis of respective rotation speeds SR and of the two successive sequences (scanning) determined according to the scanning conditions, and the above-described standby interval time Tᵢₙₜ. Details of whether or not rotation speed control is to be performed in step S102 will be described later (FIG. 3 or the like). A determination result, is held in the RAM of the system control device 124.

Next, the system control device 124 determines whether or not a scanning starting operation can be performed by the operator before main scanning is started (that is, whether or not an instruction for scanning starting is set to be permitted by operating a scanning starting button or the like) (step S103), and, in a case where the scanning starting operation is set to be permitted (step S103; Yes), the system control device 124 proceeds to step S104 and waits for a scanning starting operation to be input. In a case where a scanning starting operation is not received (step S103; No) (that is, in a case where the next sequence (scanning) is set to be automatically started after a predetermined inter-sequence delay time elapses from finishing of the previous sequence), the flow proceeds to steps S105 and S106.

In step S104, in a case where a scanning starting operation is detected (step S104; Yes), the flow proceeds to steps S105 and S106. In a case where a scanning starting operation is not detected (step S104; No), standby is performed until a scanning starting operation is detected.

During the inter-sequence delay standby in step S105, in a case where it is determined that a scanning starting operation is received in step S103 (step S103; Yes), the system control device 124 waits until a preset inter-sequence delay time (a predetermined standby time period from, detection of the scanning starting operation) elapses. In a case where it is determined that a scanning starting operation cannot be performed in step S103 (step S103; No), standby is performed until a difference time between the preset inter-sequence delay time and a time from the starting time point in step S101 to the starting time point in step S105 elapses.

In step S106 of determining whether or not rotation speed control can be performed, in a case where it is determined that rotation speed control can be performed (step S106; Yes), the flow proceeds to step S107, and a rotation speed control operation is started. In a case where it is determined that rotation speed control cannot be performed (step S106; No), the flow proceeds to step S110.

In the rotation speed control operation in steps S107 to S109, the system control device 124 controls the gantry control device 108 to decelerates the rotation disk 102 to the target, low rotation speed acquired in step S102 or to a rotation stoppage state (step S107), waits for a predetermined standby time Tittle to elapse at the target low rotation speed or in the rotation stoppage state (step S108), and then increases a rotation speed to the rotation speed Sₘ main scanning set for the next sequence (step S109).

The system control device 124 increases the rotation speed to the rotation speed Sₘ in the next sequence until a standby starting time point, of the next sequence, then, controls the gantry control device 108, the bed control device 109, and the X-ray control device 110 on the basis of the scanning conditions at the standby starting time point, and performs standby for executing the next scanning (sequence) (step S110).

After step S105 and step S110 are completed, that is, during the standby for the inter-sequence delay time, the system control device 124 reduces a rotation speed of the scanner or stops the scanner, and performs standby for a predetermined standby time in that state. Thereafter, if an increase of a rotation speed to a rotation speed in the next sequence is completed, the system control device 124 controls the X-ray control device 110 and the bed control device 109 so as to perform main scanning (step S111).

The system control device 124 determines whether or not main scanning (sequence) has been performed by the number of set sequences (step S112). If all sequences are not completed (step S112; No), the flow returns to step S101. If all of the sequences have been performed (step S112; Yes), a series of sequences illustrated in the flowchart in FIG. 2 is finished.

Next, with reference to a timing chart of FIG. 3, a description will be made of specific examples of the determination of whether or not rotation speed control is to be performed and the rotation speed control operation in step S102.

In a rotation speed control operation example illustrated in FIG. 3, the system control device 124 reduces a speed of the rotation disk 102 to a rotation stoppage state, then maintains the rotation stoppage state for a predetermined standby time, and then accelerates the rotation disk 102 to a rotation speed set for the next sequence.

In the determination of whether or not rotation speed control is to be performed (step S102), first, the system control device 124 obtains the standby interval time T_{Int}. The system control device obtains the time (deceleration time T₁) required to reduce a rotation speed S₂ (a rotation speed in the previous scanning) before rotation speed control is started to a rotation stoppage state, and the time (acceleration time T₂) required to increase a rotation speed to a rotation speed S₃ set for main scanning in the next sequence from the rotation stoppage state. The standby time T_{Idle} in the rotation stoppage state is obtained on the basis of the standby interval time T_{Int}, the deceleration time T₁, and the acceleration time T₂ (Equation (1)).

T_{Idle}=T_{Int}−(T₁+T₂) (1)

The deceleration time T₁ and the acceleration time T₂ are fixed values which are defined on the basis of the rotation speed Sₙ before rotation speed control (before deceleration) and the rotation speed Sₘset for main scanning in the next sequence. In the example illustrated in FIG. 3, n=2, and m=3.

In a case where the standby time T_{Idle} in the rotation stoppage state is equal to or more than a predetermined lower limit, value Tₜₕ (T_{Idle}=T_{Int}−(T₁+T₂)>Tₜₕ), the system control device 124 determines that rotation speed control can be performed, and starts a rotation speed control operation according to the following procedures A1 to A4.

Procedures of the rotation speed control will be described in correlation with the respective steps in the flowchart in FIG. 2.

A1) In step S106, rotation speed control is made possible.

A2) In step S107, deceleration from the rotation speed Sₙ (S₂ in FIG. 3) of the rotation disk 102 to a rotation stoppage state is started.

A3) In step S108, standby is performed until the standby time T_{Idle} elapses.

A4) In step S109, acceleration from the rotation stoppage state to the rotation speed Sₘ (S₃ in FIG. 3) for main scanning in the next sequence is started.

In a case where the standby time T_{Idle} in the rotation stoppage state is less than a predetermined lower limit value Tₜₕ (T_{Idle}=T_{Int}−(T₁+T₂)<Tₜₕ), the system control device 124 starts a process in the following B1.

B1) It is determined that rotation speed control cannot be performed in step S106, and the flow proceeds to step S110.

A rotation speed of the rotation disk 102 is maintained to be the original rotation speed Sₙ until step S110.

As described above, in the first embodiment, in a case where a standby time from finishing of the previous scanning to starting of the next main scanning is equal to or more than the predetermined time Tₜₕ, rotation of the rotation disk 102 can be stopped for a time period which is equal to or more than the predetermined lower limit value Tₜₕ. Therefore, it is possible to reduce wearing of the bearing of the rotation disk 102 compared with a case where high speed rotation is normally maintained. Since such rotation speed control is performed for the standby time (inter-sequence delay time) between scanning operations, acceleration of the rotation disk 102 is completed by starting of the next scanning, and thus examination throughput is improved. Since rotation noise is not generated during stoppage of rotation, an object or an operator can spend scanning standby time more comfortably.

Standby maybe performed in a deceleration rotation state at a predetermined reduced rotation speed (target low rotation speed) instead of a rotation stoppage state. In other words, a deceleration rotation state may be maintained after a rotation speed is reduced to a target low rotation speed, and then a rotation speed control operation including acceleration to a rotation speed set. for the next, scanning may be performed. In this case, in the determination of whether or not rotation speed control is to be performed in step S102, a standby interval time between a starting time point of the inter-sequence delay time and the next scanning standby starting time point, is obtained, a standby time in a deceleration rotation state is obtained on the basis of the deceleration time T₁, the acceleration time T₂, and the standby interval time T_{Int}, and it is determined that, rotation speed control can be performed in a case where the obtained standby time in a deceleration rotation state is equal to or more than the predetermined lower limit, value Tₜₕ.

### Second Embodiment

In a rotation speed control operation, deceleration to a rotation speed which is as low as possible may be performed even if deceleration to rotation stoppage or a target low rotation speed is not performed. In the second embodiment, the system control device 124 performs a rotation speed control operation of decelerating the rotation disk 102 to a rotation speed (hereinafter, referred to as the “minimum rotation speed”) which is as low as possible for an inter-sequence delay time, then, maintaining the deceleration rotation state for the predetermined standby time T_{Idle}, and then increasing a rotation speed to a rotation speed set for main scanning in the next sequence.

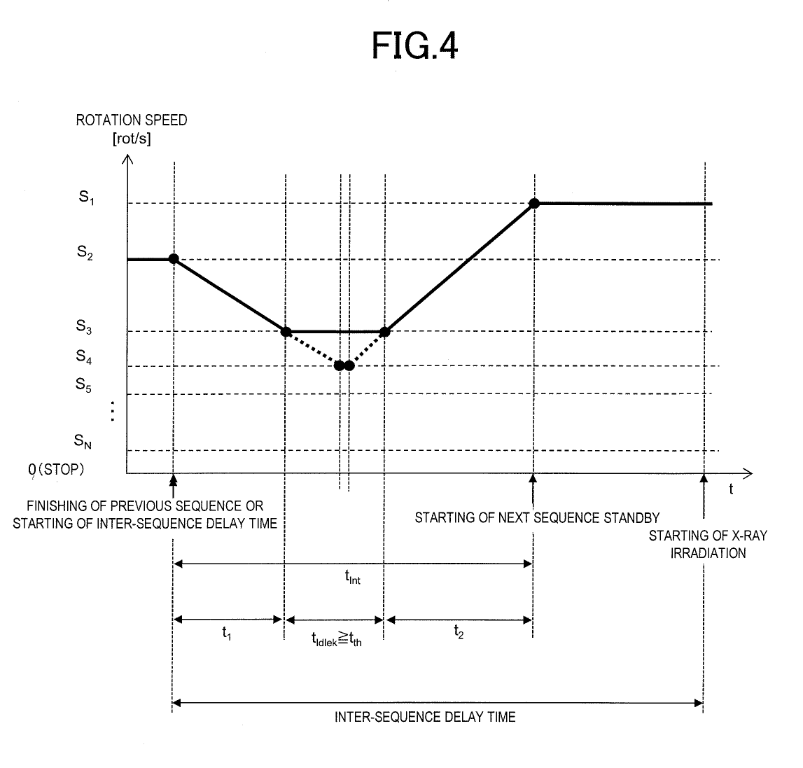

With reference to a timing chart in FIG. 4, a description will be made of a rotation speed control operation according to the second embodiment. An operation example illustrated in the timing chart in FIG. 4 is a specific example of the rotation speed control operation shown in step S107 to step S109 in FIG. 2. The same portions as those in the first embodiment are given the same reference numerals, and a repeated description will be omitted.

In the rotation speed control operation example illustrated in FIG. 4, the system control device 124 decelerates the rotation disk 102 from the rotation speed S₂ in the previous sequence (scanning) for the inter-sequence delay time, maintains rotation of the rotation disk at as low a speed as possible (the minimum rotation speed Sₙ′=S₃) for the predetermined standby time T_{Idle}, and then accelerates the rotation disk to the rotation speed S₁ set for main scanning in the next sequence (an operation example indicated by a solid line in FIG. 4).

In the timing chart in FIG. 4, a portion indicated by a dashed line shows an operation example in a case where a rotation speed is reduced to a target low rotation speed S₄ which is set in advance. In the operation example corresponding to the dashed line, if a rotation speed is reduced to the target low rotation speed S₄, a deceleration rotation state cannot be maintained for a predetermined lower limit value (Tₜₕ) or more.

In the second embodiment, the system control device 124 obtains the minimum rotation speed Sₙ′ at which a deceleration rotation state can be maintained for the predetermined lower limit, value (tₜₕ) or more. For the inter-sequence delay time, a rotation speed is reduced to the minimum rotation speed Sₙ′, then a deceleration state is maintained, and then a rotation speed is increased to a rotation speed set for main scanning in the next sequence.

In the determination of whether or not rotation, speed control is to be performed (step S102 in FIG. 2), in the same manner as in the first, embodiment, a standby interval time between a starting time point of the inter-sequence delay time and the next scanning standby starting time point may be obtained, a standby time in a deceleration rotation state may be obtained on the basis of a deceleration time T₁ to a target low rotation speed, an acceleration time T₂ which is the time required for acceleration to a rotation speed in the next sequence from the target low rotation speed, and a standby interval time t_{Int}, and it may be determined that rotation speed control can be performed in a case where the obtained standby time in a deceleration rotation state is equal to or more than the predetermined lower limit value tₜₕ.

In other words, first, the system control device 124 obtains a standby interval time t_{Int} which is a period of time between a starting time point (a finishing time point of the previous sequence in FIG. 4) of the inter-sequence delay time and the next scanning standby starting time point. The system control device obtains the time (deceleration time T₁) required to reduce a rotation speed S₂ before rotation, speed control is started to a target low rotation speed (S₄ in FIG. 4), and the time (acceleration time T₂) required to increase the target low rotation speed S₄ to a rotation speed S₁ set in the next sequence. A standby time t_{Idle} in a deceleration state is obtained on the basis of the standby interval time t_{Int}, the deceleration time T₁, and the acceleration time T₂ (Equation (2)).

t_{Idle}=t_{Int}−(T₁+T₂) (2)

Here, the deceleration time T₁ and the acceleration time T₂ are fixed values which are defined on the basis of the target low rotation speed set in advance, the rotation speed Sₙ before rotation speed control (before deceleration), and the rotation speed Sₘ set for main scanning in the next sequence. For example, in the example illustrated in FIG. 4, n=2, and m=1.

Here, the system control device 124 obtains the minimum rotation speed (Sₙ′) at which the standby time t_{Idle} in a deceleration state is equal to or more than a predetermined lower limit value tₜₕ (t_{Idle}=t_{Int}−(t₁+t₂)≧tₜₕ).

Here, t₁ is a deceleration, time required to reduce a rotation speed S₂ before rotation speed control is started, to the minimum rotation speed Sₙ′, and t₂ is an acceleration time required to increase the minimum rotation speed Sₙ′ to a rotation speed S₁ set in the next sequence.

In a case where the minimum rotation speed (Sₙ′) is present, the system control device 124 starts a rotation speed control operation according to the following procedures C1 to C4.

Procedures of the rotation speed control will be described in correlation with the respective steps in the flowchart in FIG. 2.

C1) In step S106, rotation speed control is made possible.

C2) In step S107, deceleration from the rotation speed Sₙ (S₂ in FIG. 4) to the minimum rotation speed Sₙ′ (S₃in FIG. 4) is started.

C3) In step S108, standby is performed until the standby time t_{Idle} elapses.

C4) In step S109, a rotation speed of the rotation disk 102 starts to be increased from the minimum rotation speed Sₙ′ to the rotation speed Sₘ (S₁ in FIG. 4) for main scanning in the next sequence.

In a case where the minimum rotation speed Sₙ′ is Sₙ (a rotation speed in the previous scanning), or the minimum rotation speed Sₙ′ is not present (t_{Idle}=t_{Int}−(t₁+t₂)<tₜₕ), the system control device 124 starts a process in the following D1.

D1) Rotation speed control is made impossible in step S106, and the flow proceeds to step S110.

A rotation speed of the rotation disk 102 is maintained to be the original rotation speed Sₙ until step S110.

As described above, in the second embodiment, it is possible to reduce a rotation speed to as low a speed as possible (minimum rotation speed Sₙ′) according to a set value of the inter-sequence delay time. Consequently, for example, even in a case where a rotation speed in the previous sequence is high, and thus the time required for deceleration to a rotation stoppage state is increased, deceleration to a lower rotation speed can be performed and be maintained for a predetermined time (t_{Int}) or more even if deceleration to rotation stoppage or a predefined target low rotation speed is not performed. Therefore, it is possible to reduce wearing of the bearing or generated rotation noise compared with a case where high speed rotation is normally maintained.

### Third Embodiment

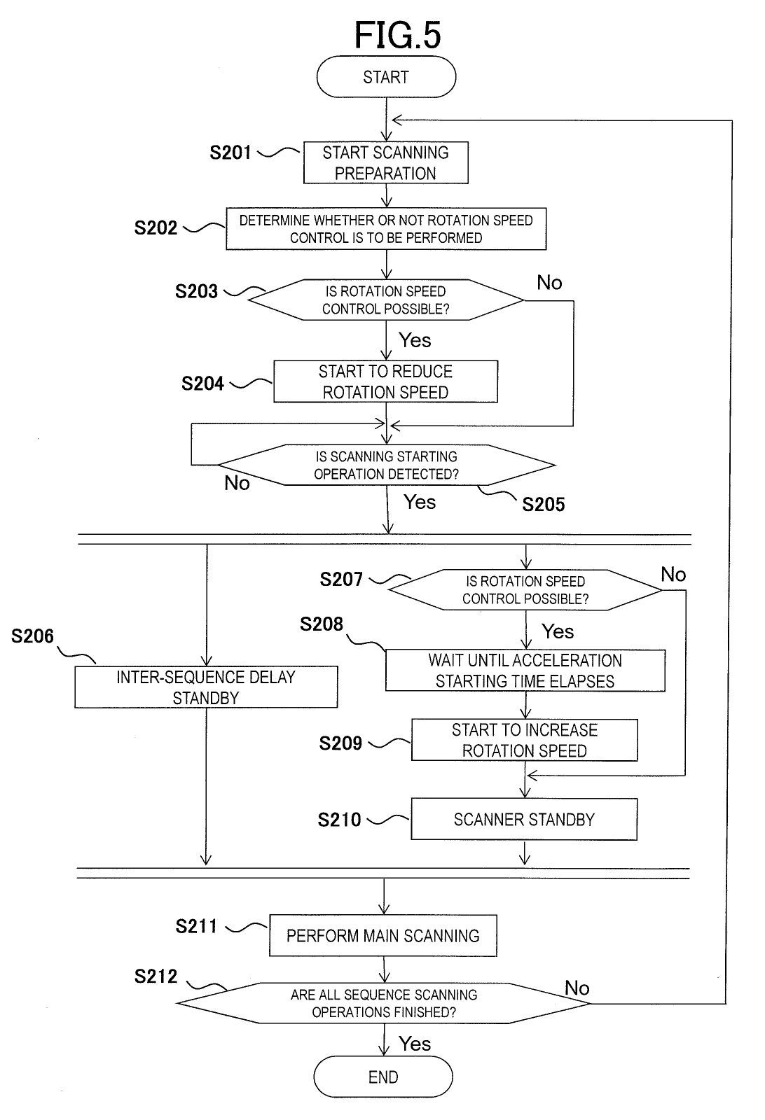

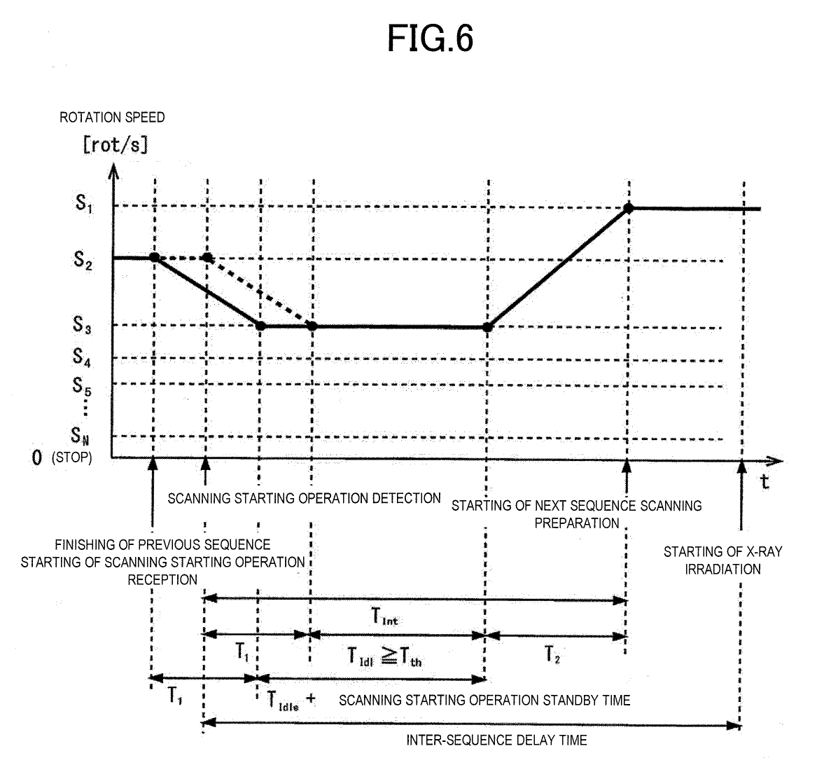

Next, a description will be made of a third embodiment, with reference to FIGS. 5 and 6.

In the first and second embodiments, in a case where a scanning starting operation is set to be received, the system control device 124 detects a scanning operation and then starts a rotation speed control operation. In other words, an indefinite standby time (a standby time until an operator performs a scanning starting operation) is generated until standby is started for the inter-sequence delay time.

In the third embodiment, in a case where a scanning starting operation is set to be permitted to be performed by an operator, the system control device 124 starts a rotation speed control operation for the rotation disk 102 in a period of waiting for a scanning starting operation.

Hereinafter, with reference to FIGS. 5 and 6, a description will be made of a rotation speed control operation according to the third embodiment.

In the same manner as in the first, and second embodiments, first, the system control device 124 starts scanning preparation (step S201).

Next, the system control device 124 determines whether or not rotation speed control is to be performed (step S202). In step S202, the system control device 124 sets a time period (hereinafter, referred to as an acceleration starting time period) from a deceleration starting time point to an acceleration starting time point with respect to a rotation speed of the rotation disk 102. The deceleration starting time point is not the rotation speed deceleration starting time point in step S204, and is the standby starting time point of the inter-sequence delay time in step S206 and the starting time point in step S207 (that is, a time point at which a scanning starting operation is detected) in the same manner as in the first and second embodiments.

The system control device 124 proceeds to step S204 if rotation speed control can be performed, and proceeds to step S205 if rotation speed control cannot be performed, on the basis of a result, of determining whether or not rotation speed control is to be performed in step S202 (step S203).

As a result of determining whether or not rotation speed control is to be performed in step S202, if rotation speed control can be performed (step S203; Yes), the system control device 124 controls the gantry control device 108 so as to start control for reducing a rotation speed of the rotation disk 102 to the target low rotation speed set in step S202 or stopping the rotation disk even before a scanning starting operation is detected (that is, the inter-sequence delay time is started) (step S204).

The system control device 124 waits for a scanning starting operation to be detected (step S205; No), and starts the inter-sequence delay time (step S206) if a scanning starting operation is detected (step S205; Yes), Step S206 is the same as step S105 in FIG. 2.

As a result of determining whether or not rotation speed control is to be performed in step S202, if rotation speed control cannot be performed (step S203; No), the system control device 124 does not reduce a rotation speed, and waits for a scanning starting operation to be detected (step S205; No), If a scanning starting operation is detected (step S205; Yes), the system control device starts the inter-sequence delay time (step S206).

In a determination process in step S207, in a case where it is determined that rotation speed control can be performed in the determination of whether or not rotation speed control is to be performed in step S202 (step S207; Yes), the flow proceeds to step S208, and a rotation speed control operation is started. In a case where it is determined that rotation speed control cannot be perforated in the determination of whether or not rotation speed control is to be performed in step S202 (step S207; No), the flow proceeds to step S210.

In a case where rotation speed control can be performed (step S207; Yes), the system control device 124 waits until the “acceleration starting time” set in step S202 elapses. The rotation disk 102 is accelerated to the rotation speed in the next sequence after the acceleration starting time elapses (step S209).

Processes in step S210 and the subsequent steps are the same as the processes in step S110 and the subsequent steps in FIG. 2. In other words, the system control device 124 increases the rotation speed to the rotation speed Sₘ in the next sequence until a standby starting time point of the next sequence, then, controls the gantry control device 108, the bed control device 109, and the X-ray control device 110 on the basis of the scanning conditions at the standby starting time point, and performs standby for executing the next scanning (sequence) (step S210).

After step S206 and step S210 are completed, that is, during the standby for the inter-sequence delay time, the system, control device 124 reduces a rotation speed of the scanner or stops the scanner, and performs standby for a predetermined standby time in that state. Thereafter, if an increase of a rotation speed to a rotation speed in the next sequence is completed,, the system control device 124 controls the X-ray control device 110 and the bed control device 109 so as to perform main scanning ( step S211).

The system control device 124 determines whether or not main scanning (sequence) has been performed by the number of set sequences (step S212). If all sequences are not completed (step S212; No), the flow returns to step S201. If all of the sequences have been performed (step S212; Yes), a series of sequences illustrated in the flowchart, in FIG. 5 is finished.

Next, with reference to a timing chart of FIG. 6, a description will be made of the rotation speed control operation in the third embodiment. A method of determining whether or not rotation speed control is to be performed is the same as that in the first embodiment.

A timing chart indicated by a solid line in FIG. 6 corresponds to a rotation speed control operation example according to the third embodiment. In FIG. 6, for comparison, the rotation speed control operation example according to the second embodiment is indicated by a dashed line.

In a case where a scanning starting operation is set to be permitted to be performed by an operator, the inter-sequence delay time T_{Int} is started from a time point at which a scanning starting operation such as pressing of a scanning starting button is detected. In the third embodiment, deceleration of the rotation disk 102 is started after a time point at which the previous sequence is finished and before detection of a scanning starting operation (before starting of the inter-sequence delay time). A standby time (indefinite) for a scanning starting operation is started from a time point, at which the previous sequence is finished.

In this case, a state in which a rotation speed is reduced can be maintained for the scanning starting operation standby time. Therefore, in the third embodiment, it is possible to further reduce wearing of the bearing, and also to further reduce rotation noise.

As described above, with reference to the accompanying drawings, preferred embodiments of an X-ray CT apparatus and the like according to the invention have been described, but the invention is not limited to these examples. It is obvious that a person skilled in the art can conceive of various modifications or alterations within the scope of the technical spirit disclosed in the present application, and it is understood that they are naturally included in the technical scope of the invention.

### REFERENCE SIGNS LIST

1 X-RAY CT APPARATUS, 100 SCANNER, 101 X-RAY SOURCE, 102 ROTATION DISK, 104 OPENING, 105 BED, 106 X-RAY DETECTOR, 107 DATA COLLECTING DEVICE, 120 OPERATION CONSOLE, 121 INPUT DEVICE, 122 IMAGE CALCULATION DEVICE, 123 STORAGE DEVICE, 124 SYSTEM CONTROL DEVICE, 125 DISPLAY DEVICE, T1 AND t1 DECELERATION TIME, T₂ AND t₂ ACCELERATION TIME, T_{Int} AND t_{Int} INTER-SEQUENCE DELAY TIME, T_{Idl} AND t_{Idl} STANDBY TIME IN DECELERATION ROTATION OR ROTATION STOPPAGE STATE, Tₜₕ AND tₜₕ LOWER LIMIT VALUE OF STANDBY TIME IN DECELERATION ROTATION OR ROTATION STOPPAGE STATE

1. An X-ray CT apparatus comprising:
a scanner that includes an X-ray source which irradiates an object with X-rays, an X-ray detector which is disposed to oppose the X-ray source and detects X-rays transmitted through the object, and a rotation disk that is mounted with the X-ray source and the X-ray detector and is rotated around the object; and
a controller that controls a rotation speed of the rotation disk for a standby time to be lower than all rotation speeds in successive scanning operations on the basis of the standby time between the successive scanning operations and the times required for deceleration and acceleration of the rotation disk.

2. The X-ray CT apparatus according to claim 1,
wherein the controller performs a rotation speed control operation of performing deceleration to a rotation stoppage state, then maintaining the rotation stoppage state, and then performing acceleration to a rotation speed set for the next scanning.

3. The X-ray CT apparatus according to claim 2,
wherein the controller
obtains a standby interval time which is a period of time between a starting time point of an inter-sequence delay time and a standby starting time point of the next scanning, and
obtains a standby time in the rotation stoppage state on the basis of a deceleration time which is the time required for deceleration to the rotation stoppage state from a rotation speed in the previous scanning, an acceleration time which is the time required for acceleration to a rotation speed in the next scanning from the rotation stoppage state, and the standby interval time, and performs the rotation speed control operation in a case where the obtained standby time in the rotation stoppage state is equal to or more than a predetermined lower limit value.

4. The X-ray CT apparatus according to claim 1,
wherein the controller performs a rotation speed control
operation of performing deceleration to a target low rotation speed, then maintaining a deceleration rotation state, arid then performing acceleration to a rotation speed set for the next scanning.

5. The X-ray CT apparatus according to claim 4,
wherein the controller
obtains a standby interval time which is a period of time between a starting time point of an inter-sequence delay time and a standby starting time point of the next scanning, and
obtains a standby time in the deceleration rotation state on the basis of a deceleration time which is the time required for deceleration to the deceleration, rotation state at a target low rotation speed from a rotation speed in the previous scanning, an acceleration time which is the time required for acceleration to a rotation speed in the next scanning from the deceleration rotation state, and the standby interval time, and performs the rotation speed control operation in a case where the obtained standby time in the deceleration rotation state is equal to or more than a predetermined lower limit value.

6. The X-ray CT apparatus according to claim 4,
wherein the controller
obtains minimum rotation speed at which a standby time in the deceleration rotation state is equal to or more than a predetermined lower limit value in the rotation speed control operation, and
performs the rotation speed control operation with the obtained minimum rotation speed as the target low rotation speed in a case where the minimum rotation speed is present.

7. The X-ray CT apparatus according to claim 2,
wherein the controller starts the rotation speed control operation after a time point at which a scanning starting operation is detected in a case where an operator is permitted to perform a scanning starting operation, or starts the rotation speed control operation after a time point at which the previous scanning is finished in a case where the operator is not permitted to perform the scanning starting operation.

8. The X-ray CT apparatus according to claim 2,
wherein, in a case where an operator is permitted to perform a scanning starting operation, the controller starts the rotation speed control operation after a time point at which the previous scanning is finished and before detection of the scanning starting operation performed by the operator.

9. A scanning method for an X-ray CT apparatus including an X-ray source which irradiates an object, with X-rays, an X-ray detector which is disposed to oppose the X-ray source and detects X-rays transmitted through the object, and a rotation disk that is mounted with the X-ray source and the X-ray detector and is rotated around the object, the method comprising:
causing a controller of the X-ray CT apparatus to control a rotation speed of the rotation disk for a standby time to be lower than all rotation speeds in successive scanning operations on the basis of the standby time between the successive scanning operations and the times required for deceleration and acceleration of the rotation disk.
